(19) United States
(12) Patent Application Publication
(10) Pub. No.: US 2015/0062320 A1
(43) Pub. Date: Mar. 5, 2015

(54) DIFFUSE REFLECTANCE HYPERSPECTRAL IMAGING SYSTEM

(71) Applicant: Board of Regents, The University of Texas System, Austin, TX (US)

(72) Inventor: James W. Tunnell, Austin, TX (US)

(21) Appl. No.: 14/478,616

(22) Filed: Sep. 5, 2014

Related U.S. Application Data
(60) Provisional application No. 61/874,139, filed on Sep. 5, 2013.

(51) Int. Cl. G01N 21/27; G01N 33/483; H04N 5/235; G01J 3/28

(52) U.S. Cl. CPC G01N 21/27; G01J 3/2823; G01N 33/4833; H04N 5/2354; G01N 2201/0683; G01N 2201/068
USPC 348/77; 356/300

(57) ABSTRACT

An imaging apparatus comprises a first optical fiber configured to deliver a source beam of light generated by a light source and a polarizing beam splitter configured to polarize the source beam of light and direct diffusely reflected light toward a collection fiber optically coupled to an optical detector. The imaging apparatus also comprises a mirror configured to reflect the polarized beam of light onto the lens and direct reflected light away from a lens. The lens is configured to focus the polarized source beam of light onto an area of sample material and focus diffusely reflected light from the sample material into a reflection beam. The imaging apparatus also comprises a plano-convex curvature matching window disposed at a focal plane of the lens, wherein a convex surface of the curvature matching window is substantially matched to the focal plane curvature of the imaging lens.

### CROSS-REFERENCE TO RELATED APPLICATIONS

This application claims the benefit of U.S. Provisional Application No. 61/874,139, filed Sep. 5, 2013, the disclosure of which is herein incorporated by reference in its entirety.

### GOVERNMENT GRANT INFORMATION

This invention was made with government support under Grant Number CA132032 awarded by the National Institutes of Health and in part with a state grant from the Advanced Research Program from the Texas Higher Education Coordinating Board. The government has certain rights in the invention.

### TECHNICAL FIELD

This application relates generally to diffuse reflectance spectroscopy for measuring optical and physiological properties of skin tissues and, more particularly, to a diffuse reflectance spectral imaging (DSRi) system that is configured to acquire wide field hyperspectral images of tissue.

### BACKGROUND

Existing non-invasive imaging device technologies, such as Melafind® and SIAscope® can only acquire images of qualitative contrast between images and are unable to provide quantitative measurements of optical scattering and absorption in a tissue sample. This lack of quantitative data regarding scattering and absorption means that existing technologies are unable to quantify the concentration or amount of a chromophore (a chemical group capable of selective light absorption resulting in the coloration of certain organic compounds) or identify biological microstructures in universally meaningful units.

This means that existing non-invasive technologies have limited utility in many areas which would benefit from quantitative imaging, such as diagnostic applications, including cancer screening, or other applications, such verifying the effectiveness of tattoo removal, planning treatment parameters for tattoo removal, measuring nanoparticle depositions, cosmetic evaluation, wound healing response (such as burns), evaluation of skin disorders, or any application that requires assessing blood content, blood oxygenation, and other chromophore content.

As a result, invasive techniques must be used in order to gather the necessary quantitative data for such applications. For example, the current early detection of skin cancers relies on a critical macroscopic visual analysis of the changes in the cutaneous lesions. Suspected malignancies are excised and analyzed using standard histopathology for diagnosis and treatment decisions.

The most widely used technique to determine whether or not a skin lesion is malignant is a simple biopsy. The problems with this technique are that it is painful and takes five to seven days to receive results. Both pain-free detection and quick results are important to customers. Another problem with this technique is that the decision to biopsy varies greatly with the experience of a dermatologist. Dermatologists are also only likely to biopsy lesions they believe may be malignant and do not have the time to biopsy all skin lesions. Therefore, there is a possibility that malignant skin lesions will go undetected.

Additionally, diagnostic accuracy for the current clinical examination is inherently qualitative and depends largely on the experience of the physician. It has been shown that general practitioners often have a much lower diagnostic accuracy than expert dermatologists. In addition, access to dermatologists can be limited by geography, financial barriers, and a shortage of supply. Second, the majority of cutaneous melanoma arise in atypical nevi which can easily go unnoticed because they appear as standard moles. In addition, for patients with familial and/or dysplastic nevus syndrome (>100 nevi), it is impossible to excise all suspected dysplastic nevi. Finally, although the sensitivity for the detection of melanoma has been improving (70-90%), the specificity is still quite low, resulting in a large number of unnecessary biopsies which increases costs and morbidity of the procedure. Therefore, a non-invasive method to inspect these lesions would be of great clinical importance.

Currently, when nonmelanoma skin cancers are removed, the surgeon is required to take an excess margin of skin around the lesion to account for nonclinically relevant spread of the tumor. This excess margin can result in a larger scar and greater cosmetic and functional deformity. Noninvasive techniques for limiting the size of these surgical excisions would potentially spare patients from requiring expensive grafting and reconstruction procedures. Indeed, with one in five Americans developing skin cancer in their lifetime, there is a growing need to equip healthcare professionals with a tool that will provide quantitative as well as qualitative images that can improve on the subjectivity of skin cancer screenings.

### SUMMARY

According to one aspect, the present disclosure is direct to an imaging apparatus comprising a first optical fiber configured to deliver a source beam of light generated by a light source and a polarizing beam splitter configured to polarize the source beam of light and direct diffusely reflected light toward a collection fiber optically coupled to an optical detector. The imaging apparatus may also comprise a mirror configured to reflect the polarized beam of light onto the lens and direct reflected light away from a lens. The lens may be configured to focus the polarized source beam of light onto an area of sample material and focus diffusely reflected light from the sample material into a reflection beam. The imaging apparatus may also comprise a plano-convex curvature matching window disposed at a focal plane of the lens, wherein a convex surface of the curvature matching window is substantially matched to the focal plane curvature of the imaging lens.

In accordance with another aspect, the present disclosure is directed to an imaging system that comprises a first optical fiber configured to deliver a source beam of light generated by a light source and a polarizing beam splitter, optically coupled to the first optical fiber, and configured to polarize the source beam of light. The imaging apparatus may also comprise a mirror configured to reflect the polarized beam of light onto a lens. The lens may be configured to focus the polarized source beam of light onto an area of sample material. The imaging apparatus may also comprise a plano-convex curvature matching window disposed at a focal plane of the lens, wherein a convex surface of the curvature matching window is substantially matched to the focal plane curvature of the imaging lens. The imaging apparatus may also comprise a second optical fiber configured to receive diffusely reflected light from the sample material.

### BRIEF DESCRIPTION OF THE DRAWINGS

FIG. 1 illustrates multiple views of a detector fiber that can be utilized with the diffuse reflectance hyperspectral imaging system according to an exemplary embodiment;

FIG. 2 illustrates an example of single point tissue sampling and photon migration through a tissue sample;

FIG. 3 shows a comparison between wide field imaging and point scanning imaging;

FIG. 4 is a diagram of an apparatus for diffuse reflectance hyperspectral imaging according to an exemplary embodiment;

FIGS. 5A-5B illustrate multiple views of the apparatus for diffuse reflectance hyperspectral imaging according to an exemplary embodiment;

FIG. 6 illustrates additional views of the apparatus for diffuse reflectance hyperspectral imaging according to an exemplary embodiment;

FIG. 7 is component diagram of the apparatus for diffuse reflectance hyperspectral imaging with additional features according to an exemplary embodiment;

FIG. 8 illustrates a flowchart for diffuse reflectance hyperspectral imaging according an exemplary embodiment;

FIG. 9 illustrates a reflectance spectra fitting curve;

FIG. 10 illustrates normalization images and equations which can be utilized with the system for diffuse reflectance hyperspectral imaging according to an exemplary embodiment;

FIG. 11 illustrates a comparison of a curvature matching window with a flat window and source-collector separation;

FIG. 12 illustrates the resolution of an image produced by the system for diffuse reflectance hyperspectral imaging according an exemplary embodiment;

FIG. 13 illustrates the measurement of sampling geometry according to an exemplary embodiment;

FIG. 14 illustrates best fit curves for optical property fitting parameters;

FIG. 15 illustrates optical property maps produced by the system for diffuse reflectance hyperspectral imaging according to an exemplary embodiment;

FIG. 16 illustrates additional property maps produced by the system for diffuse reflectance hyperspectral imaging according to an exemplary embodiment;

FIG. 17 illustrates hyperspectral images of a mole produced by the system for diffuse reflectance hyperspectral imaging according to an exemplary embodiment;

FIG. 18 illustrates an overview of the technique for generating a hyper-spectral image cube of a sample according to an exemplary embodiment; and

FIG. 19 illustrates an exemplary computing environment that is part of the system for diffuse reflectance hyperspectral imaging according to an exemplary embodiment.

### DETAILED DESCRIPTION

The embodiments described herein provide a non-invasive device and system which can be used to acquire quantitatively meaningful images of tissue and measure spatially resolved quantities of biochemical or morphological agents. Furthermore, certain embodiments described herein describe an imaging device and system which can provide quantitative results in a short period of time.

This application is related to application Ser. No. 13/029,992 (U.S. Patent Application Publication No. 2012/0057145), titled “SYSTEMS AND METHODS FOR DIAGNOSIS OF EPITHELIAL LESIONS,” filed Feb. 17, 2011, the disclosure of which is herein incorporated by reference in its entirety.

While methods, apparatuses, and computer-readable media are described herein by way of example, those skilled in the art recognize that methods, apparatuses, and computer-readable media for imaging are not limited to the embodiments or drawings described. It should be understood that the drawings and description are not intended to be limited to the particular form disclosed. Rather, the intention is to cover all modifications, equivalents and alternatives falling within the spirit and scope of the disclosure. Any headings used herein are for organizational purposes only and are not meant to limit the scope of the description or the claims. As used herein, the word “may” is used in a permissive sense (i.e., meaning having the potential to) rather than the mandatory sense (i.e., meaning must). Similarly, the words “include,” “including,” and “includes” mean including, but not limited to.

Methods, apparatuses and computer-readable media are described for imaging samples, such as skin samples, non-invasively and in a short period of time. The systems and devices disclosed herein allows clinicians to image skin samples using a handheld device to which is used to generate quantitative images of tissue structures. The resulting images can be used for diagnostic purposes, such as diagnostic applications, including cancer screening, or other applications, such verifying the effectiveness of tattoo removal, planning treatment parameters for tattoo removal, measuring nanoparticle depositions, cosmetic evaluation, wound healing response (such as burns), evaluation of skin disorders, or any application that requires assessing blood content, blood oxygenation, and other chromophore content.

Reflectance spectroscopy is used to quantitatively measure the color and intensity of reflected light. Light reflected from tissue is modified by the processes of absorption and scattering. Absorption can occur at a variety of wavelengths, which depend on the structural, biological, and chemical makeup of a tissue sample. Light scattering produces changes in the trajectory of incident light and affects all wavelengths to varying degrees. Examining the wavelength of light reflected off a sample provides information about absorption properties of the sample. Additionally, examining light scattering provides information about the way that light (photons) propagates through the tissues in the sample, and therefore allows scientists and clinicians to determine details about tissue structure (e.g. cell nuclear size and shape, connective tissue organization, epithelial tissue structure/thickness, etc.).

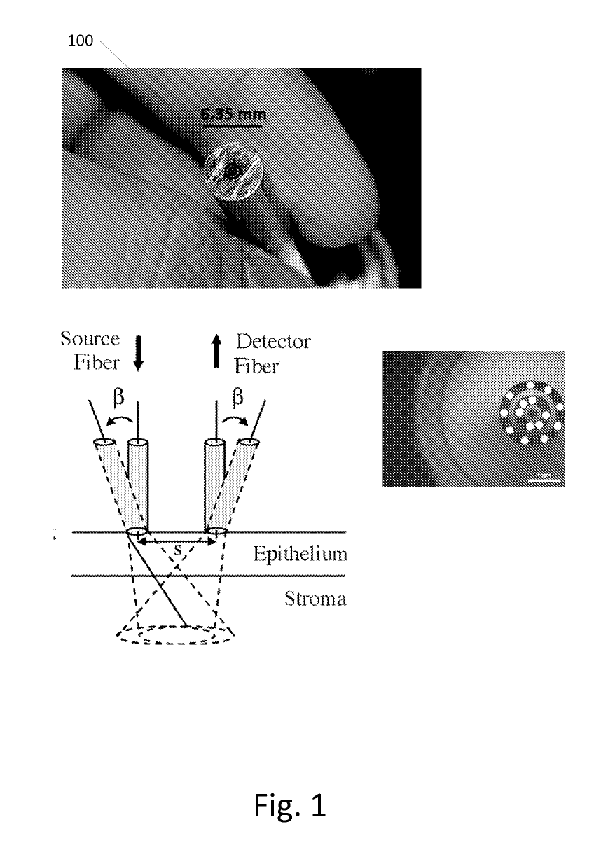

The interplay of the two processes, absorption and scattering, provides access to a great deal of diagnostic information which can be used to evaluate a tissue sample. Typically, reflectance spectroscopy systems for biomedical applications contain three key components: a light source, a fiber optic probe, and a spectrometer. FIG. 1 illustrates a light scattering spectroscopy apparatus including a source fiber and detector fiber. Light is guided down to the source fiber to illuminate the tissue, and one or a plurality of detector fibers collect light re-emitted from the tissue. The geometry of the source and collection fibers determine the path the light propagates through the tissue, and thus, can be used to control the depth and path-length of the light.

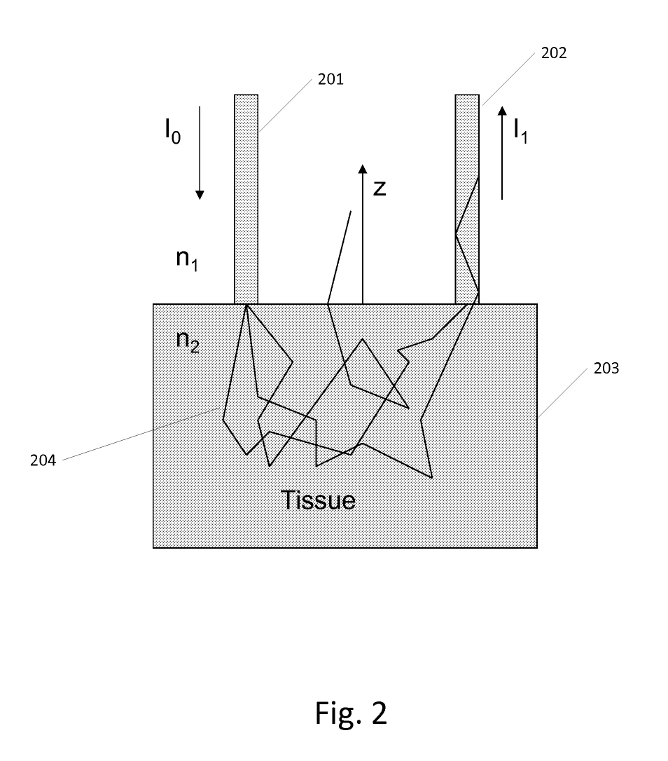

FIG. 2 illustrates the path 204 that a photon could take as it passes from a source fiber 201, through a tissue sample 203, and to a detector fiber 202. By measuring scattering of photons, the extent to which the angular path of light is altered by biological structures such as nuclei and mitochondria can be determined. Additionally, quantitative morphological information about the tissue sample can be obtained through more sophisticated analysis of the angular dependence, polarization dependence, and wavelength dependence of the scattered light.

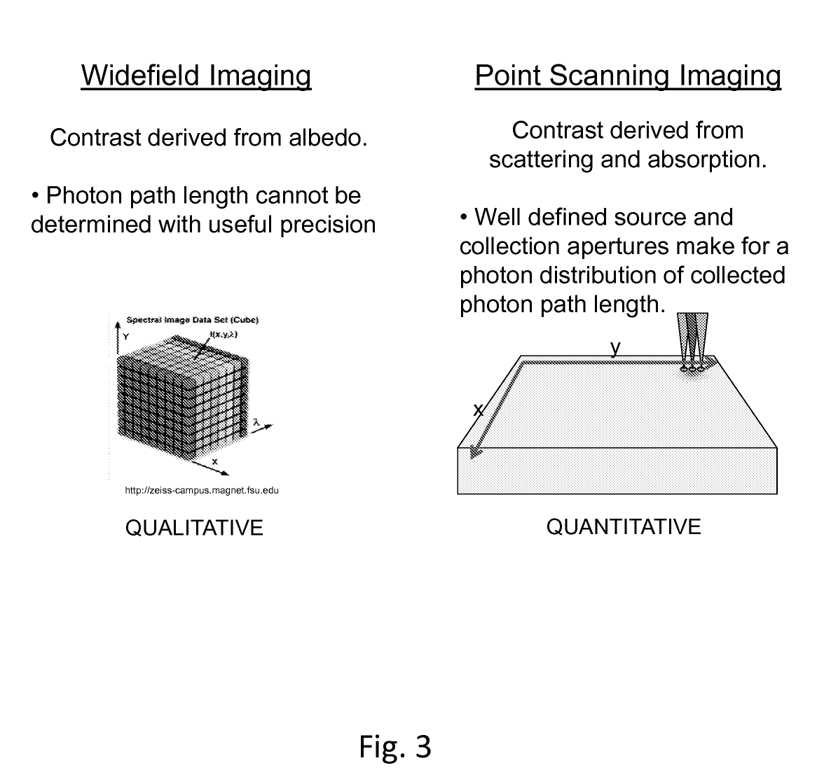

FIG. 2 illustrates an example of a point measurement which is used in point scanning imaging. Point scanning imaging has a number of advantages over widefield imaging, as illustrated in FIG. 3. In point scanning imaging, the contrast is derived from scattering and absorption, as described above. The well-defined source and collection apertures make for a photon distribution of collected photon path length. In widefield imaging, contrast is derived from albedo (percentage of reflected light) and photon path length cannot be determined with useful precision. This results in only qualitatively useful information, not quantitatively useful information.

The present system utilizes diffuse reflectance spectral imaging (DRSi), which is capable of quantitative imaging of skin and other superficial tissues (and turbid materials in general). The quantitative imaging aspect of this technique allows for the mapping of the concentrations of particular molecular species and scattering properties of a material. For example, DRSi produces images of skin blood content (in mg/ml), a particularly important feature of skin tumors that have increased blood content due to angiogenesis.

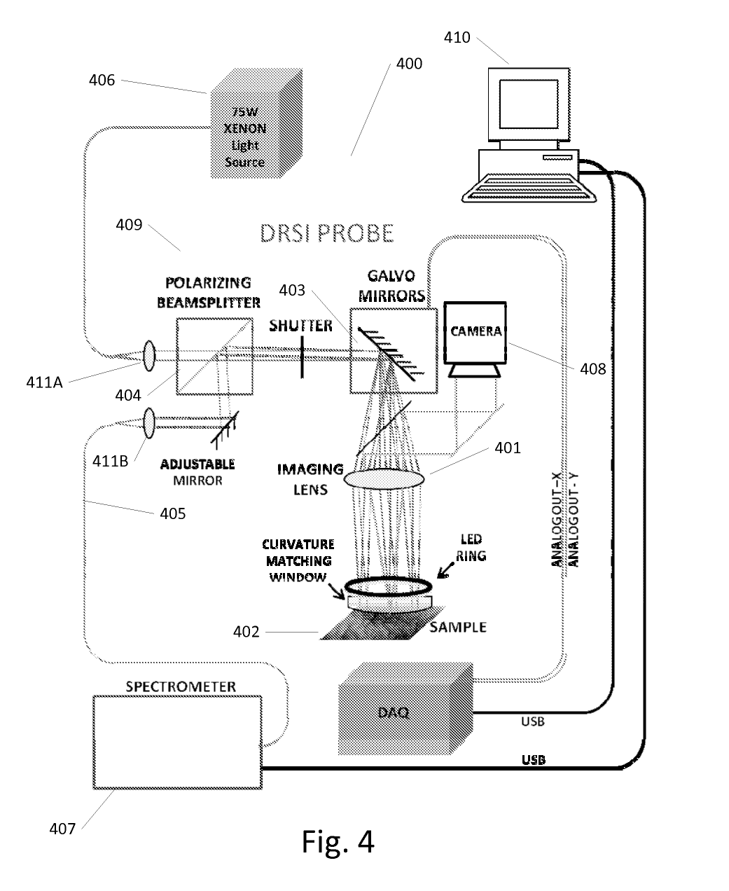

DRSi collects hyper-spectral macroscopic images of turbid (diffusely scattering) materials and can be implemented as a compact apparatus, such as a hand-held scanner device. For example, FIG. 4 illustrates an imaging apparatus 400 according to an exemplary embodiment. The apparatus includes a lens 401 configured to focus a polarized beam of light onto a point in a sample 402 and focus diffusely reflected light returned from the sample into a reflection beam, wherein the reflection beam includes a polarized component and a depolarized component.

The apparatus 400 includes a mirror 403 configured to reflect the polarized beam of light onto the lens 401 and reflect the reflection beam away from the lens 401, a polarizing beam splitter 404 configured to generate the polarized beam of light from a beam of light and to direct a portion of the de-polarized component of the reflection beam towards an optical detector 405, as well as a light source 406 configured to generate the beam of light.

As shown in FIG. 4, the light source 406 is used to generate a beam of broadband light, which passes through collimation lens 411A and is polarized using a polarizing beam splitter 404 before the light reflects off mirror 403 and is focused onto a spot on the sample 402 by the lens 401. The diffusely reflected light that emerges from the surface of the material after having undergone multiple scattering and absorption events passes through the lens 401 and is focused into a reflection beam which optically retraces the illumination path, with the exception of a deflection of depolarized light by the beam splitter into a collection arm of the system. Of course, the diffusely reflected light can also be collected at an aperture which is adjacent to the aperture through which the beam of broadband light passes.

In FIG. 4 the collection arm is shown as an adjustable mirror which reflects the light deflected from the polarizing beam splitter 404 through collimation lens 411B onto optical detector 405, which can be an optical fiber as discussed earlier. In other words, a first component of the reflection beam will pass through the polarizing beam splitter due to having the original polarization, and a second component which has become depolarized will again be polarized by the polarizing beam splitter, with a portion of the second component being directed towards the adjustable mirror and optical detector 405.

The polarizing beam splitter serves to remove specular reflections from the tissue surface. Specular reflections will preserve the polarization, and thus, when this reflected light is directed back to the beamsplitter, the specularly reflected light will pass through the beam splitter. This leaves the multiply and diffusely scattered light, which will randomize the polarization and which will be directed to the optical detector. In this way, the polarizing beam splitter picks off the orthogonal polarization of the incident light, and thus contains the signature of the diffusely scattered light.

The system can include a beam scanning device to steer the focused spot across a two dimensional field of the sample 402. For example, the mirror 403 can be a mirror galvanometer which is configured to rotate and adjust the location of the point in the sample where the polarized beam of light is focused. In this way, a spectrum can be collected to each point of scan until the entire field has been sampled.

The apparatus 400 can include a spectrometer 407 coupled to the optical detector of the reflection beam. The apparatus 400 can also include a camera 408 configured to capture an image of the diffusely reflected light returned from the sample for documentation with standard color imaging and a housing 409 enclosing the lens 401, the mirror 403, and the polarizing beam splitter 404. Additionally, the apparatus can further include a Data Acquisition (DAQ) unit coupled to the camera 408, an LED ring for illumination of the tissue surface, a curvature matching window to account for field curvature of the scanned beam, and a computing device 410 coupled to the spectrometer 407 and the DAQ.

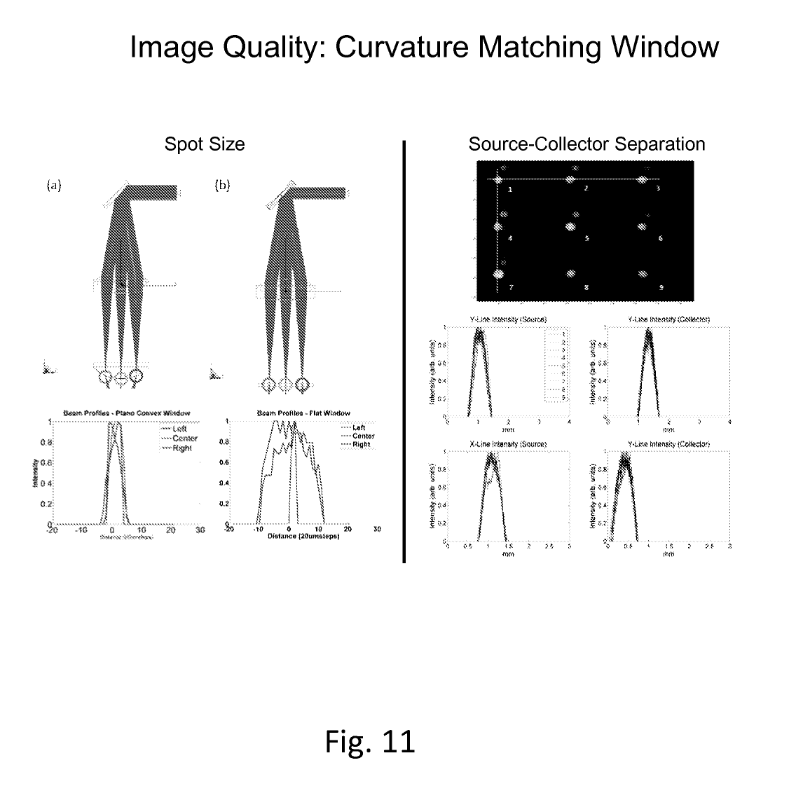

For images of homogeneous phantoms, each reflectance image pixel should ideally be the same in order for optical property maps to be accurate. If this criteria is met (within the statistical variation of the signal due only to shot and dark current noise), then any contrast within the image may be attributable only to variations within the tissue. However, in any beam scanning system, there will be artifacts and aberrations; in the case of DRSi, one of the main artifacts is field curvature. One example to mitigate field curvature effects includes the use of f-theta scan lenses that effectively reduce the effect of field curvature through a series of meniscus lenses. Another, more cost effective embodiment involves matching the field curvature to the surface of the skin to be imaged. This may be achieved through using a field curvature matching window (plano-convex lens), which was in contact with the tissue. The convex shape of the matching window provides additional functional versatility; concave skin topography (neck, back) can be imaged. This is visually illustrated in FIG. 11, where the results for TracePro simulations for planar and plano-convex matching windows are shown.

As a result of the scaled-down approach to building a beam-scanning based imaging system consistent with certain disclosed embodiments, the imaging lens focal length may be limited. This may lead to field curvature and limited depth of field. To address both of these issues, a plano-convex lens may be fixed at the focal plane such that the convex surface closely matches the focal plane curvature to ensure that the tissue surface remains in focus. Since this lens functions more as a window, it may be referred to as a “curvature matching window.” In order to select a plano-convex lens of proper curvature, TracePro imaging simulation software (Lambda Research, MA) may be used to optimize the curvature for even distribution of focal spot sizes at a large majority of the points of scan.

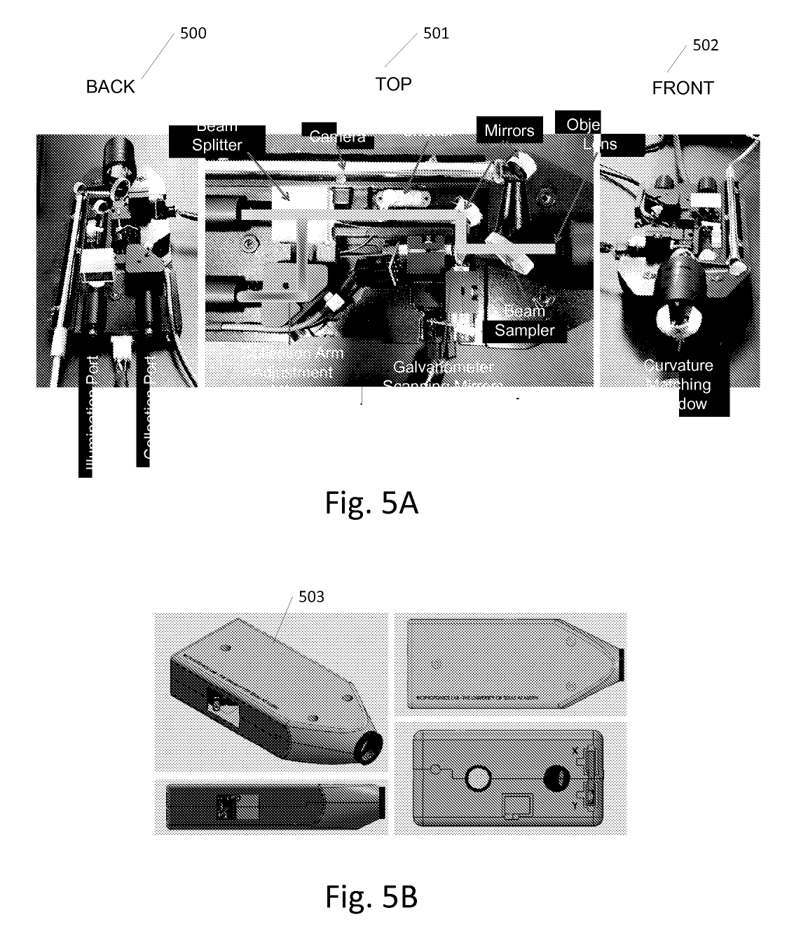

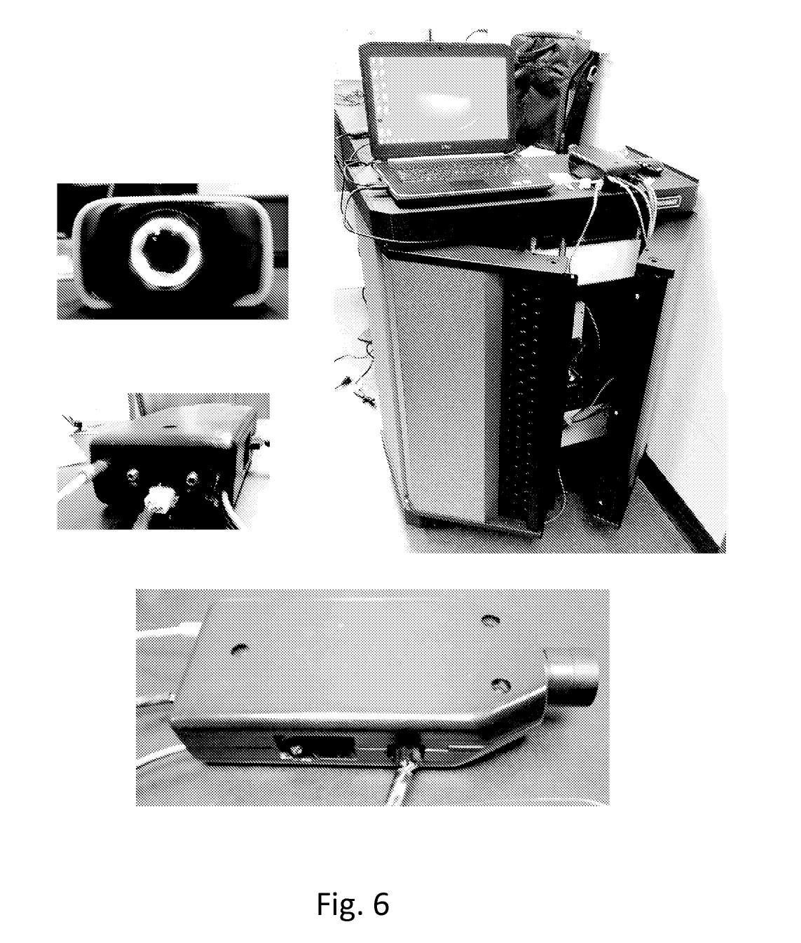

FIG. 5A illustrates additional views of the handheld device which is part of the imaging apparatus according to an exemplary embodiment, including a back view 500, top view 501, and a front view 502. FIG. 5B shows and external view of the handheld device, including housing 503. FIG. 6 shows additional views of the apparatus, including a computing workstation which can be part of the apparatus and additional external views of the handheld device which can house one or more of the apparatus components.

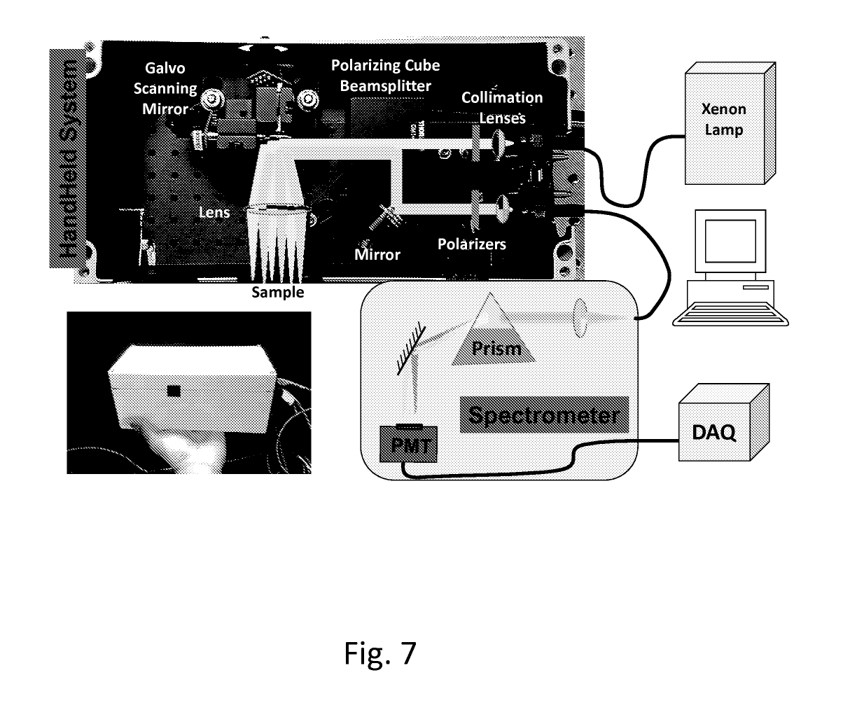

FIG. 7 illustrates another imaging apparatus according to an exemplary embodiment. The imaging apparatus of FIG. 7 includes polarizers proximate to the collimation lenses to polarize the initial beam of light and the portion of the de-polarized component of the reflection beam which is deflected by the polarizing beam splitter. Similar to the polarizing beam splitter, the polarizers can be utilized to minimize collected specular reflection.

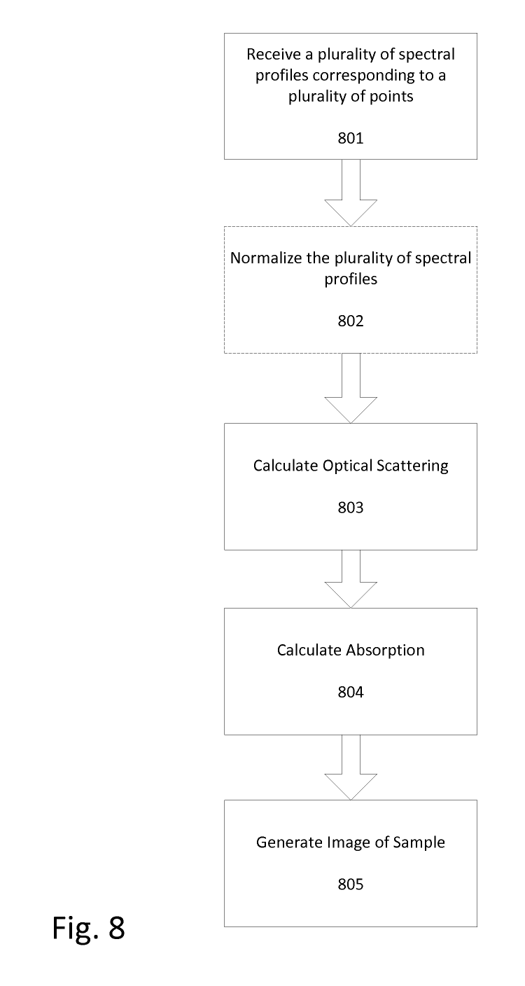

FIG. 8 illustrates a flowchart for a method of imaging according to an exemplary embodiment. At step 801a plurality of spectral profiles corresponding to a plurality of points on a sample are received. This can include spectral profiles that are generated by the spectrometer previously discussed and/or based on measurements captured by the imaging apparatus of FIG. 4 or 7. For example, each spectral profile can be generated by focusing a beam of polarized light on a corresponding point and collecting diffusely reflected light returned from the sample. Of course, a single spectral profile corresponding to a single point can also be received instead of a plurality of spectral profiles. For example, the spectral profiles corresponding to each point on a sample can be received one at a time and stored until a plurality of spectral profiles have been compiled.

At step 802, which can be optional, the plurality of spectral profiles can be normalized based on one or more factors. The normalization can be performed prior to calculating optical scattering and prior to calculating absorption for the plurality of spectral profiles. Normalization can be performed on all, none, or some subset of the plurality of spectral profiles. For example, spectral profiles that have a low signal to noise ratio, or some other metric, can be normalized, while the remaining spectral profiles remain un-normalized. The one or more factors used to normalize spectral profiles can include, for example, variations in light source, detector spectral intensity variations, detector efficiency variations, and spatial heterogeneity.

At step 803, optical scattering can be calculated, such as optical scattering of the beam of polarized light which was focused on a point in the sample. Optical scattering can be calculated for each spectral profile in the plurality of spectral profiles, or just for a subset of the plurality of spectral profiles, such as one spectral profile.

At step 804, absorption can be calculated, such as absorption of the beam of polarized light which was focused on a point in the sample. Optical scattering can be calculated for each spectral profile in the plurality of spectral profiles, or just for a subset of the plurality of spectral profiles, such as one spectral profile. Of course, absorption can be calculated prior to the calculation of optical scattering as well as afterwards, and these examples and the flowchart of FIG. 8 is not intended to be limiting with regard to order of steps or number of steps.

At step 805, an image of the sample is generated based at least in part on the calculation of optical scattering and/or the calculation of absorption. The amount of information which is gathered using the present system allows for the generation of images and calculation of properties which provide quantitative information and metrics about the sample and the structures within the sample.

Alternatively or additionally, image processing in this DRSi system may consist of background subtraction followed by spatial and spectral normalization. The image background may consist of back reflections from optics in the system and any constant stray light that travels into the collection arm. According to at least one embodiment, the background may be acquired as an image of a highly absorbing, non-scattering material in contact with the curvature matching window and may be subtracted from all subsequent images. Light delivery and signal collection efficiency in DRSi may be a function of the imaging lens' numerical aperture (NA) and, to a lesser degree, of the scan mirror size and position. The scan mirror size affects the NA as it dictates the diameter of the collection f-stop. Spatial intensity heterogeneities due to scan angle effects may be accounted for by imaging a homogeneous sample used to normalize these effects. For this purpose, a PDMS sample infused with titanium dioxide (TiO2) for scattering may be used as a reflectance standard to spatially and spectrally normalize subsequent tissue images. The following equations may be used to describe the culmination of these normalization operations that result in reflectance images.

$$R ( x , y , λ ) = \frac{( {I}_{Tissue} ( x , y , λ ) - {I}_{Bkg} ( x , y , λ ) )}{( {I}_{Standard} ( x , y , λ ) - {I}_{Bkg} ( x , y , λ ) )}$$

The spectral reflectance of TiO2 is invariant with wavelength, which provides a reference that accounts for any spatial and spectral heterogeneities across the field of view (FOV). The TiO2 reflectance value was calculated as follows:

$${R}_{{TiO}_{2}} = \frac{( {I}_{{TiO}_{2} - {I}_{Bkg}} )}{( {I}_{Spectralon} - {I}_{Bkg} )}$$

The reflectance of the TiO2 phantom as a standard measurement may be calculated by comparing its diffuse reflectance to that of a known NIST traceable reflectance standard (e.g., Spectralon, Labsphere Inc.).

There are multiple ways to glean optical and physiological properties from a diffuse reflectance spectrum. According to one embodiment, a look-up table (LUT) method may be used. The LUT is essentially a database of reflectance spectra for all (or at least a large majority of) physiologically relevant absorption (μₐ) and scattering (μ′ₛ) coefficients. The LUT may be numerically fitted to measured reflectance spectra from skin in order to extract μₐ and μₛ′. The LUT method to may also be used obtain optical property maps of reduced scattering (μₛ′ at 630 nm), blood volume fraction and melanin content.

At the image fitting stage, the LUT may be numerically fit to the diffuse reflectance spectrum of each pixel, yielding optical property values for that particular pixel. The fitting may be performed using custom algorithms written in MATLAB that employ non-linear fitting techniques to minimize the error between the LUT and the measured data. With the data combined from all the pixels, a two-dimensional map may be obtained for each optical property. The fitting step may be performed offline, as reflectance spectra typically takes about one second per spectra to fit; although several methods exist to speed this process up several orders of magnitude.

The present systems and devices can be used to image superficial biological tissues in order to visualize quantified spatial distributions of biologically intrinsic or extrinsic chromophores including but not limited to, oxy/deoxyhemoglobin, eu/pheomelanin, water, beta carotene, bilirubin, nanoparticles and/or tattoo inks.

This present systems and devices can also be used to visualize the spatially resolved characteristics of tissue microstructure and metabolism such as mean cell size, mean cell nuclear size, collagen, nicotinamide adenine dinucleotide (NADH) and flavin adenine dinucleotide (FAD) concentration.

DRSi captures hyper-spectral images using a point scanning technique of a wideband light source. Because light is focused to a single point and light is collected from a single point nearby, the optical sampling is spatially confined as opposed to other approaches. This spatial confinement makes it possible to measure optical scattering and absorption independently and quantify the concentration of chromophores and scattering properties. Without spatial confinement, the collected reflectance intensity would be a sole function of albedo, a combination of scattering and absorption. Spatial confinement allows for the control of the optical path length, and thus, the ability to quantify optical absorption and scattering. Due to the ability to quantify the concentration of chromophores, system functionality can easily be extended to fluorescence hyperspectral imaging, where the influences of blood and melanin absorption can be accounted for and removed from collagen, NADH and FAD fluorescence images.

The images produced by the system can include one or more chomophores in the sample that were determined based at least in part on the optical scattering and the absorption. The one or more chromophores can include oxyhemoglobin, deoxyhemoglobin, eumelanin, pheomelanin, water, beta carotene, bilirubin, nanoparticles, and tattoo inks.

The images produced by the system can include one or more spatially resolved characteristics of tissue in the sample that were determined based at least in part on the optical scattering and the absorption. The one or more spatially resolved characteristics can include mean cell size, mean cell nuclear size, collagen, nicotinamide adenine dinucleotide concentration, and flavin adenine dinucleotide concentration.

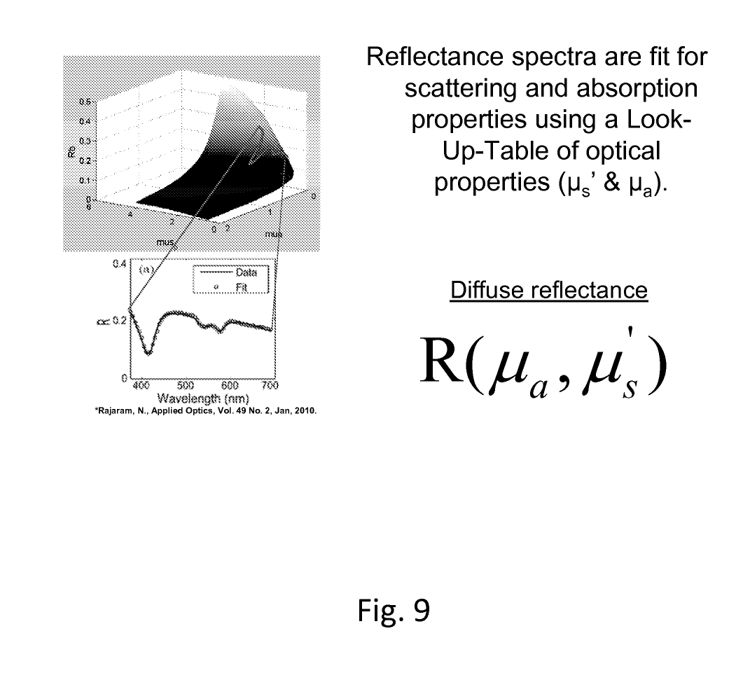

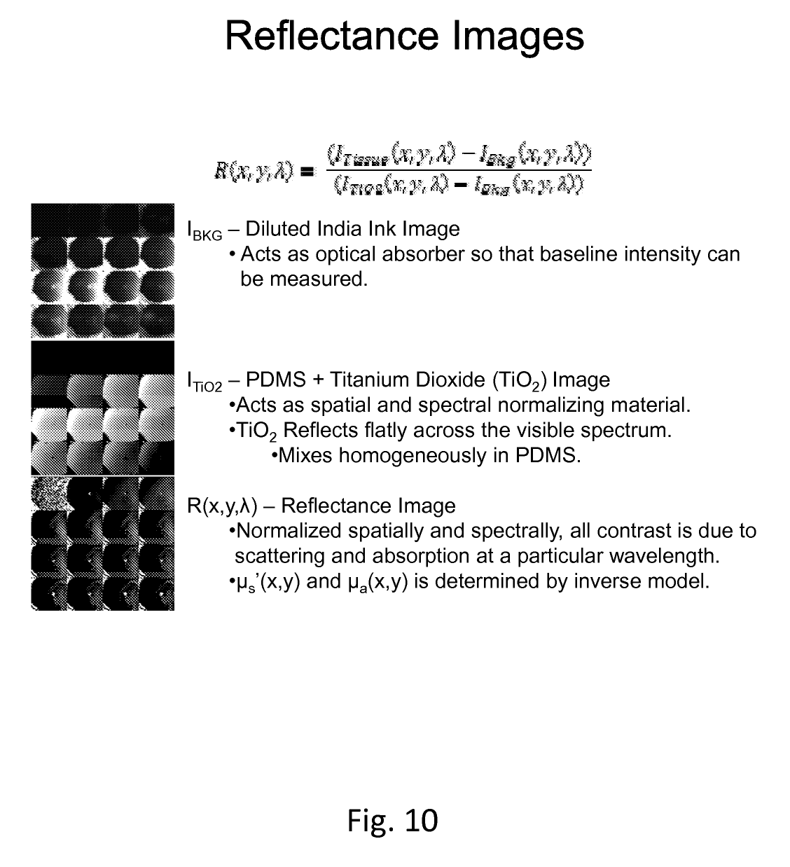

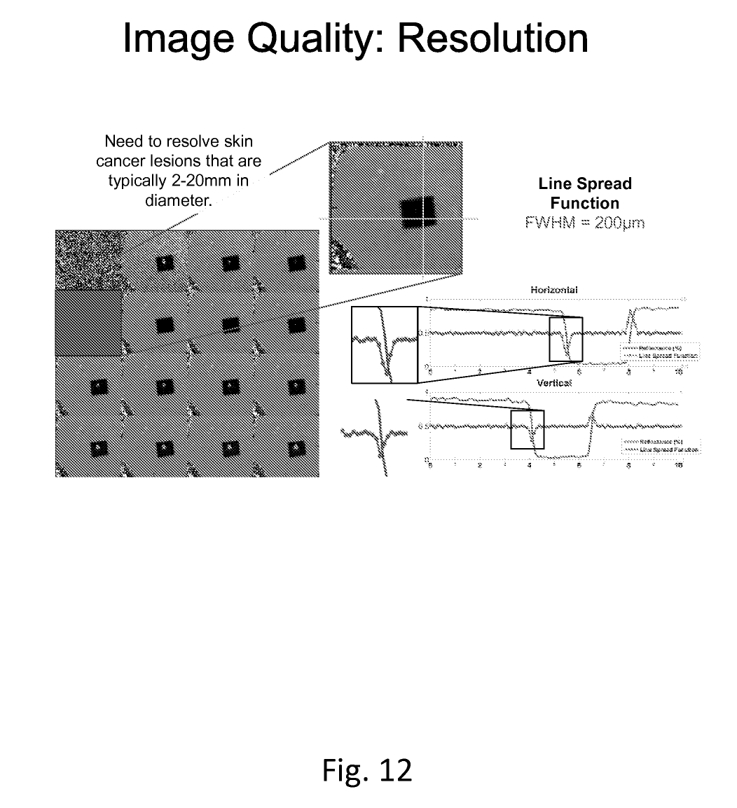

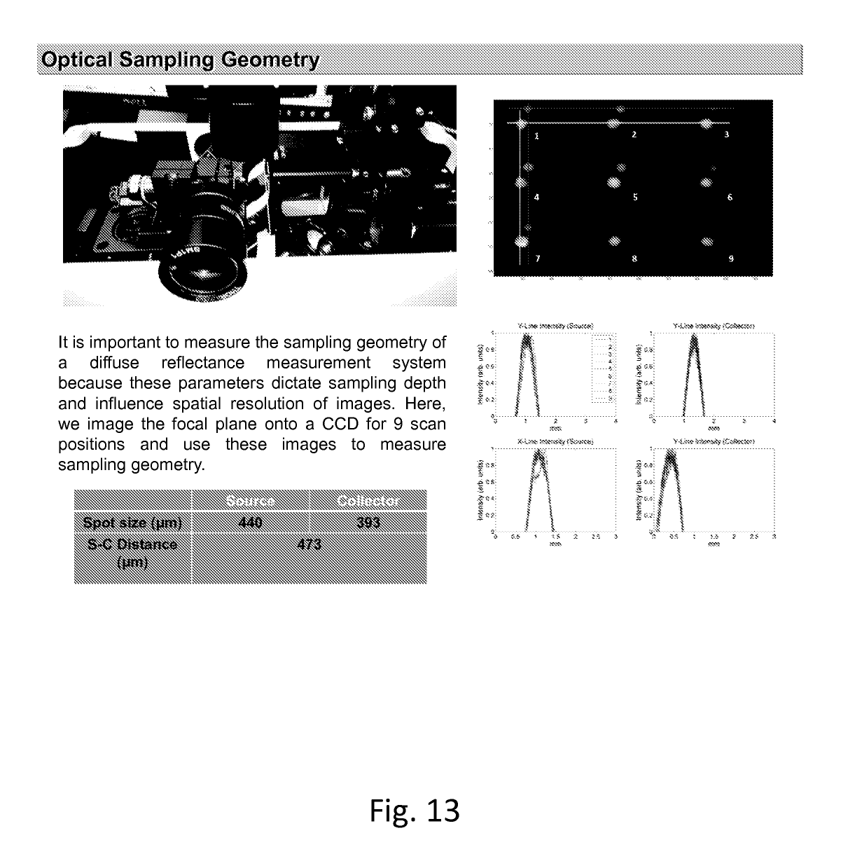

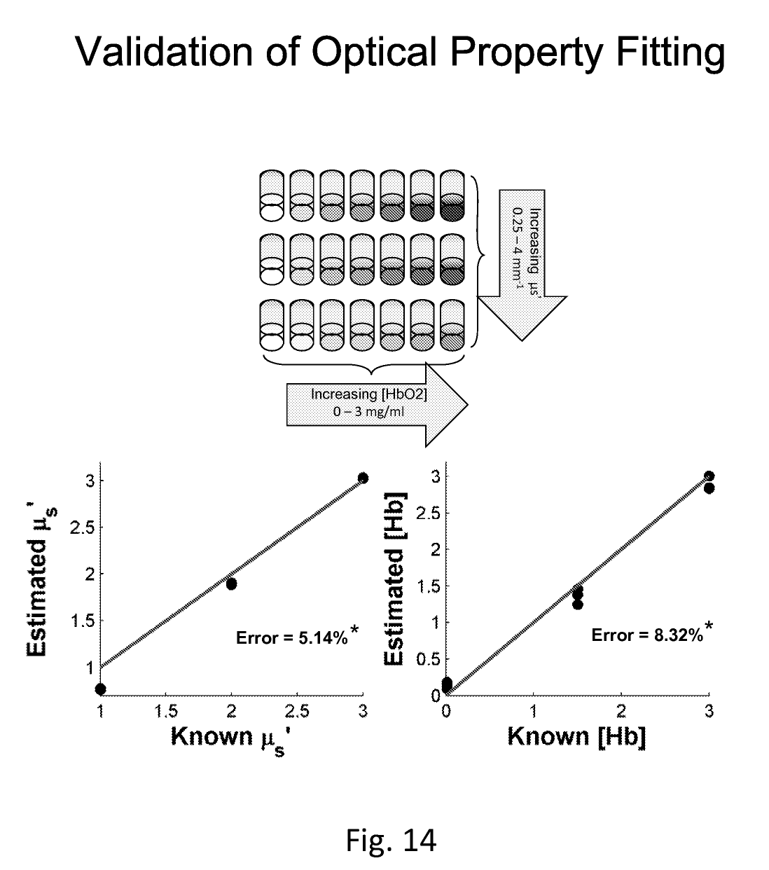

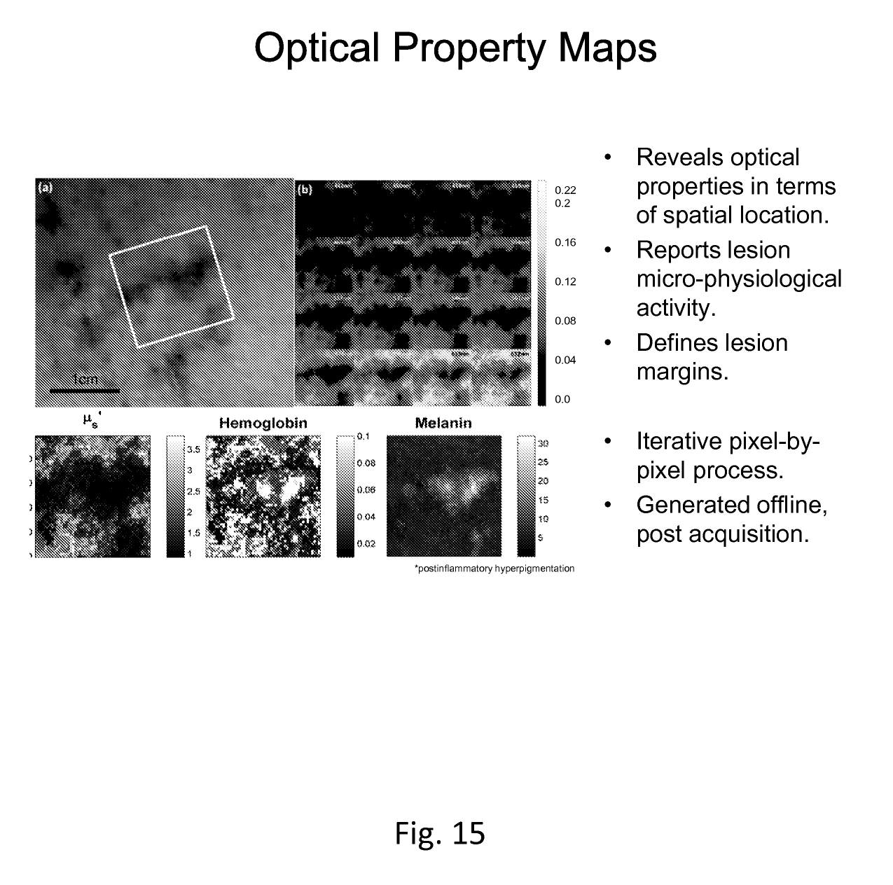

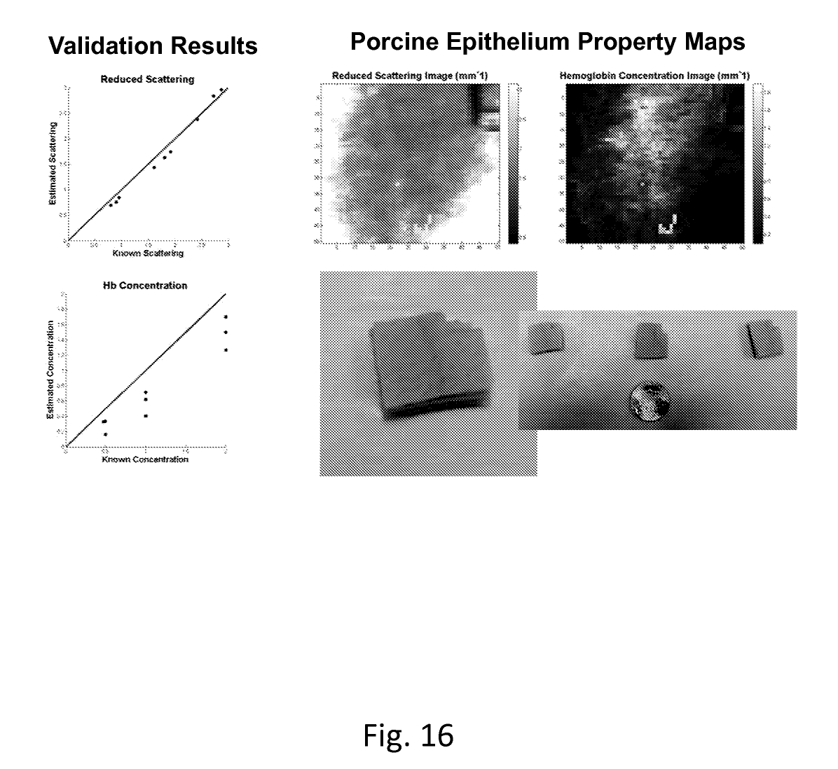

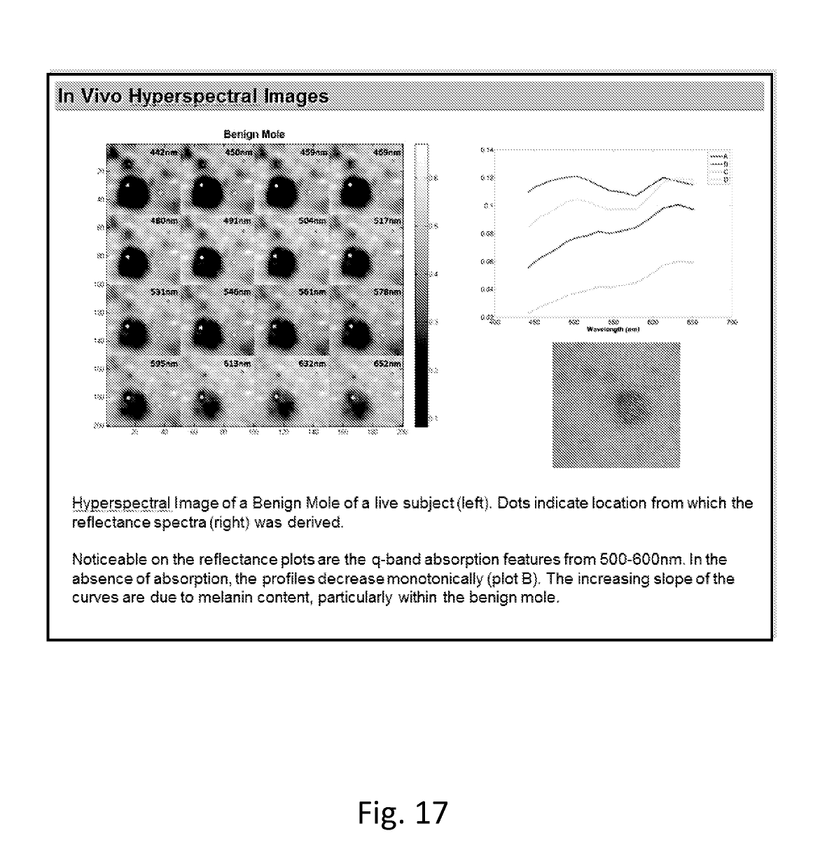

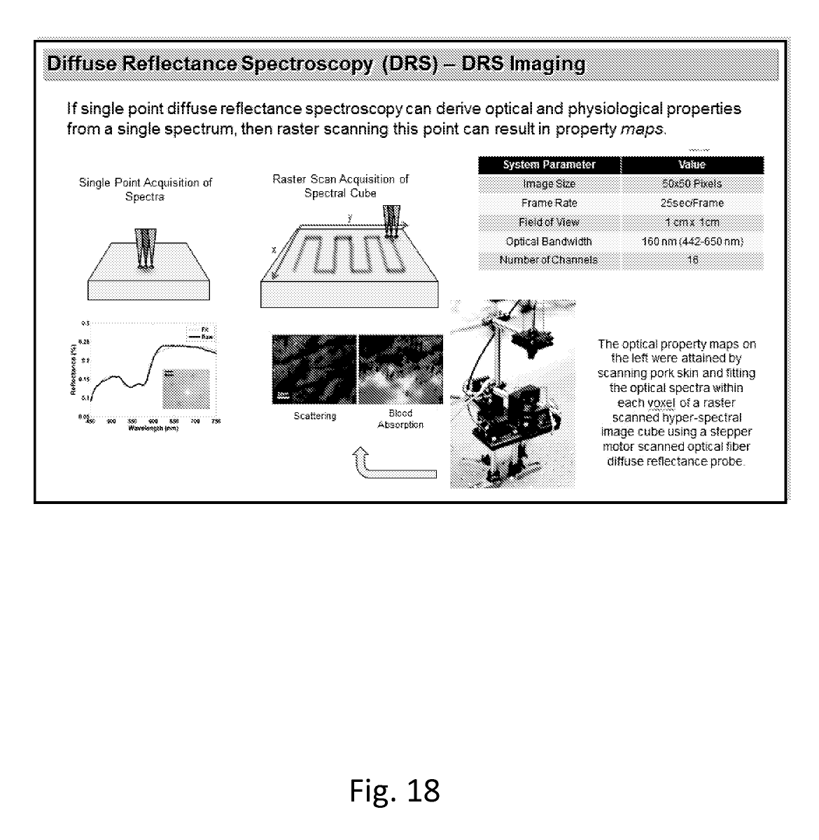

FIGS. 9-18 provide additional details of the system, examples of spectral calculations, and resulting property maps, including fitting curves for reflectance spectra based on absorption and scattering, normalization equations and images, image quality with regard to the curvature matching window, image resolution requirements, optical sampling geometry, validation of optical property fitting, optical property maps, validation results and property maps, hyperspectral images produced from in-vivo measurements, and system parameters than can be used with the imaging system. FIG. 11 illustrates a graph showing the system's ability to maintain the source and collector geometry over the full field of view. Maintaining this geometry allows for precise control of the optical path length throughout the imaging field.

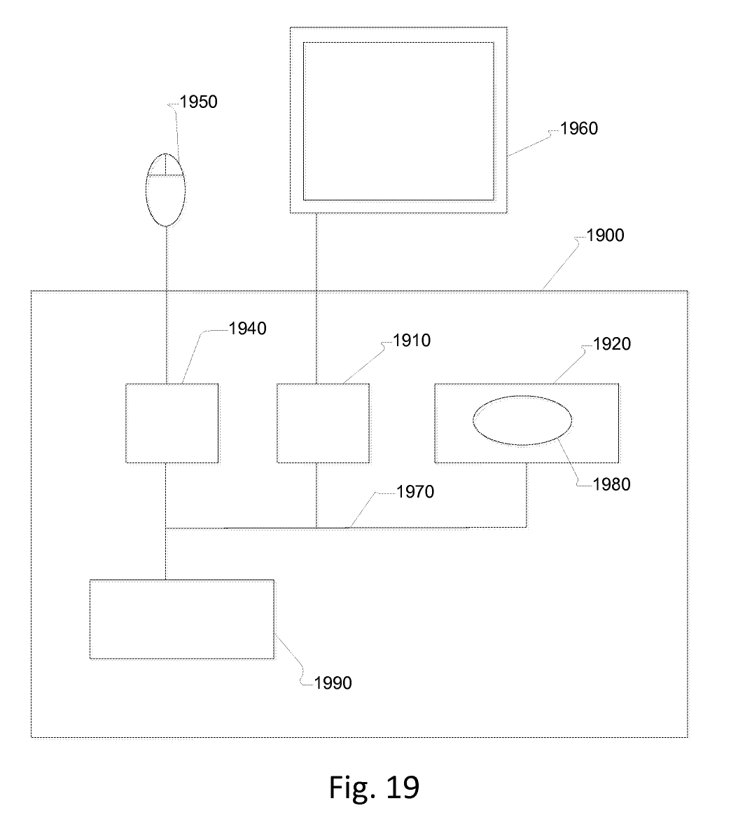

One or more of the above-described techniques can be implemented in or involve one or more computer systems. FIG. 19 illustrates a generalized example of a computing environment 1900. The computing environment 1900 is not intended to suggest any limitation as to scope of use or functionality of a described embodiment.

With reference to FIG. 19, the computing environment 1900 includes at least one processing unit 1910 and memory 1920. The processing unit 1910 executes computer-executable instructions and may be a real or a virtual processor. In a multi-processing system, multiple processing units execute computer-executable instructions to increase processing power. The memory 1920 may be volatile memory (e.g., registers, cache, RAM), non-volatile memory (e.g., ROM, EEPROM, flash memory, etc.), or some combination of the two. The memory 1920 may store software instructions 1980 for implementing the described techniques when executed by one or more processors. Memory 1920 can be one memory device or multiple memory devices.

A computing environment may have additional features. For example, the computing environment 1900 includes storage 1940, one or more input devices 1950, one or more output devices 1960, and one or more communication connections 1990. An interconnection mechanism 1970, such as a bus, controller, or network interconnects the components of the computing environment 1900. Typically, operating system software or firmware (not shown) provides an operating environment for other software executing in the computing environment 1900, and coordinates activities of the components of the computing environment 1900.

The storage 1940 may be removable or non-removable, and includes magnetic disks, magnetic tapes or cassettes, CD-ROMs, CD-RWs, DVDs, or any other medium which can be used to store information and which can be accessed within the computing environment 1900. The storage 1940 may store instructions for the software 1980.

The input device(s) 1950 may be a touch input device such as a keyboard, mouse, pen, trackball, touch screen, or game controller, a voice input device, a scanning device, a digital camera, remote control, or another device that provides input to the computing environment 1900. The output device(s) 1960 may be a display, television, monitor, printer, speaker, or another device that provides output from the computing environment 1900.

The communication connection(s) 1990 enable communication over a communication medium to another computing entity. The communication medium conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

Implementations can be described in the general context of computer-readable media. Computer-readable media are any available media that can be accessed within a computing environment. By way of example, and not limitation, within the computing environment 1900, computer-readable media include memory 1920, storage 1940, communication media, and combinations of any of the above.

Of course, FIG. 19 illustrates computing environment 1900, display device 1960, and input device 1950 as separate devices for ease of identification only. Computing environment 1900, display device 1960, and input device 1950 may be separate devices (e.g., a personal computer connected by wires to a monitor and mouse), may be integrated in a single device (e.g., a mobile device with a touch-display, such as a smartphone or a tablet), or any combination of devices (e.g., a computing device operatively coupled to a touch-screen display device, a plurality of computing devices attached to a single display device and input device, etc.). Computing environment 1900 may be a set-top box, mobile device, personal computer, or one or more servers, for example a farm of networked servers, a clustered server environment, or a cloud network of computing devices.

Having described and illustrated the principles of our invention with reference to the described embodiment, it will be recognized that the described embodiment can be modified in arrangement and detail without departing from such principles. It should be understood that the programs, processes, or methods described herein are not related or limited to any particular type of computing environment, unless indicated otherwise. Various types of general purpose or specialized computing environments may be used with or perform operations in accordance with the teachings described herein. Elements of the described embodiment shown in software may be implemented in hardware and vice versa.

Although described in the context of skin and tissue, the system described herein can be utilized with any sample for which the imaging techniques would be useful. In view of the many possible embodiments to which the principles of our invention may be applied, we claim as our invention all such embodiments as may come within the scope and spirit of the following claims and equivalents thereto.

What is claimed is:

1. An imaging apparatus comprising:
a first optical fiber configured to deliver a source beam of light generated by a light source;
a polarizing beam splitter configured to:
polarize the source beam of light; and
direct diffusely reflected light toward a collection fiber optically coupled to an optical detector;
a mirror configured to:
reflect the polarized beam of light onto the lens; and
direct reflected light away from a lens, wherein the lens is configured to:
focus the polarized source beam of light onto an area of sample material; and
focus diffusely reflected light from the sample material into a reflection beam; and
a plano-convex curvature matching window disposed at a focal plane of the lens, wherein a convex surface of the curvature matching window is substantially matched to the focal plane curvature of the imaging lens.

2. The imaging apparatus of claim 1, further comprising:
a camera configured to capture an image of the sample material;
an LED ring, disposed between the lens and the plano convex curvature matching window, and configured to illuminate the sample material during capturing of images by the camera; and
a shutter, disposed between the beam splitter and mirror, and configured to block light from the light source during capturing of images by the camera.

3. The imaging apparatus of claim 2, further comprising a spectrometer coupled to the optical detector and configured to generate a spectral profile of the reflection beam.

4. The imaging apparatus of claim 3, further comprising a processing system configured to associate the image of an area of the sample material captured by the image with spectral profile data of the reflection beam collected from the area of the sample material.

5. The imaging apparatus of claim 1, wherein the mirror comprises a mirror galvanometer which is configured to rotate and adjust the location of the point in the sample where the polarized beam of light is focused.

6. The imaging apparatus of claim 1, wherein the optical detector comprises a second optical fiber.

7. The imaging apparatus of claim 1, wherein the first optical fiber, the polarizing beam splitter, the mirror, the lens, and the plano-convex curvature matching window are disposed in a common housing.

8. An imaging system, comprising:
a first optical fiber configured to deliver a source beam of light generated by a light source;
a polarizing beam splitter, optically coupled to the first optical fiber, and configured to polarize the source beam of light;
a mirror configured to reflect the polarized beam of light onto a lens, wherein the lens is configured to focus the polarized source beam of light onto an area of sample material;
a plano-convex curvature matching window disposed at a focal plane of the lens, wherein a convex surface of the curvature matching window is substantially matched to the focal plane curvature of the imaging lens; and
a second optical fiber configured to receive diffusely reflected light from the sample material.

9. The imaging system of claim 8, further comprising:
a camera configured to capture an image of the area of the sample material;
an LED ring, disposed in an optical path between the lens and the plano convex curvature matching window, and configured to illuminate the sample material during capturing of images by the camera; and
a shutter, disposed in an optical path between the beam splitter and mirror, and configured to block light from the light source during capturing of images by the camera.

10. The imaging system of claim 9, further comprising a spectrometer coupled to the second optical fiber and configured to generate a spectral profile of the diffusely reflect light from the sample material.

11. The imaging system of claim 9, further comprising a processing system configured to associate the image of the area of the sample material captured by the image with spectral profile data of the diffusely reflect light from the sample material collected from the area of the sample material.

12. The imaging system of claim 8, wherein the mirror comprises a mirror galvanometer which is configured to rotate and adjust the location of the area in the sample where the polarized beam of light is focused.

13. The imaging system of claim 8, wherein the first optical fiber, the polarizing beam splitter, the mirror, the lens, and the plano-convex curvature matching window are disposed in a common housing.

14. The imaging system of claim 8, wherein the light source comprises a Xenon arc lamp.
